(19) United States
(12) Patent Application Publication
(10) Pub. No.: US 2025/0210188 A1
(43) Pub. Date: Jun. 26, 2025

(54) MEDICATION DELIVERY DEVICE WITH WAKE-UP FEATURE

(71) Applicant: Eli Lilly and Company, Indianapolis, IN (US)

(72) Inventor: Adam Nathaniel WIESLER, Zionsville, IN (US)

(21) Appl. No.: 18/850,684

(22) PCT Filed: Mar. 20, 2023
(86) PCT No.: PCT/US2023/015597

§ 371 Date: Sep. 25, 2024

Related U.S. Application Data
(60) Provisional application No. 63/323,794, filed on Mar. 25, 2022.

(51) Int. Cl. G16H 40/63; A61M 5/315; G16H 20/17

(52) U.S. Cl. CPC G16H 40/63; A61M 5/31566; G16H 20/17; A61M 2205/3306; A61M 2205/3368; A61M 2205/3584; A61M 2205/50; A61M 2205/583; A61M 2205/8212

(57) ABSTRACT

The techniques described herein relate to computerized methods and apparatus for determining when a medication delivery device is ready for use. The method includes obtaining, from a temperature sensor of a medication delivery device, temperature data indicative of a temperature. The method includes obtaining, from an ambient light sensor, ambient light data indicative of an amount of ambient light to which at least a portion of the medication delivery device is exposed. The method includes determining, based on the temperature data, whether the temperature exceeds a temperature threshold. The method includes determining, based on the ambient light data, whether the amount of light exceeds an ambient light threshold. The method includes outputting an indication that the medication delivery device is ready for use when the temperature exceeds the temperature threshold and the amount of ambient light exceeds the ambient light threshold.

### BACKGROUND OF INVENTION

Many medication products need to be stored in low temperatures. Patients are often instructed not to consume or administer these products while they are cold. Prior to consumption or use, patients therefore need to remove the medication products from cool storage and allow them to warm up for a specified duration of time.

### SUMMARY OF INVENTION

According to an exemplary embodiment of the present disclosure, a computerized method is provided. The method includes obtaining, from a temperature sensor of a medication delivery device, temperature data indicative of a temperature. The method includes obtaining, from an ambient light sensor, ambient light data indicative of an amount of ambient light to which at least a portion of the medication delivery device is exposed. The medication delivery device includes a reservoir configured to hold the medication. The medication delivery device includes an actuating button for initiating an injection of the medication. The method includes determining, based on the temperature data, whether the temperature exceeds a temperature threshold. The method includes determining, based on the ambient light data, whether the amount of light exceeds an ambient light threshold. The method includes outputting an indication that the medication delivery device is ready for use when the temperature exceeds the temperature threshold and the amount of ambient light exceeds the ambient light threshold.

In some embodiments, the method includes determining, based on the temperature data and previously-obtained temperature data, whether the temperature has increased over time. In some embodiments, outputting the indication that the medication delivery device is ready for use includes outputting the indication after determining that the temperature has increased over time.

In some embodiments, the medication delivery device includes a visual indicator. In some embodiments, outputting the indication that the medication delivery device is ready for use includes outputting the indication via the visual indicator.

In some embodiments, the visual indicator includes at least one light emitting diode (LED).

In some embodiments, obtaining the temperature data and the ambient light data includes obtaining the temperature data and the ambient light data after a specified duration of time has elapsed since an activation event.

In some embodiments, obtaining the temperature data and the ambient light data includes receiving a wireless signal. In some embodiments, obtaining the temperature data and the ambient light data includes obtaining the temperature data and the ambient light data after a specified duration of time has elapsed since receiving the wireless signal.

In some embodiments, the wireless signal includes a Bluetooth low energy (BLE) signal.

In some embodiments, obtaining the temperature data includes obtaining data indicative of a temperature of a medication held within the medication delivery device.

In some embodiments, outputting the indication that the medication delivery device is ready for use after an elapsed period of time after the temperature exceeds the temperature threshold and the amount of ambient light exceeds the ambient light threshold and after a period.

In some embodiments, providing a first warming-up indication when the temperature does not exceed the temperature threshold and/or the amount of ambient light does not exceed the ambient light threshold, providing a second warming-up indication during timing of said elapsed period of time, or both.

### BRIEF DESCRIPTION OF DRAWINGS

Additional embodiments of this disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a cross-sectional view of an injection device prior to use.

FIG. 2 is a cross-sectional view of the injection device with the syringe assembly in a storage position and ready for an injection event.

FIG. 3 is a cross-sectional view of the injection device with the syringe assembly in an injection position.

FIG. 4 is a cross-sectional view of the injection device illustrating a placement of one or more main PCBs within an end portion of the injection device's housing.

FIG. 5A is a top (i.e., a distal) perspective view of the main PCB and a secondary PCB.

FIG. 5B is a bottom (i.e., a proximal) perspective view of the main PCB and the secondary PCB.

FIG. 6A is a top view of the main PCB and the secondary PCB.

FIG. 6B is a bottom view of the main PCB and the secondary PCB.

FIG. 7 is a system architecture view of electrical components within an injection device and of an external device.

FIG. 8 is a block diagram depicting an exemplary system 800 for determining when a medication delivery device is ready for use, according to some embodiments of the technology described herein.

FIG. 9 is a flowchart showing an exemplary computerized method 900 for determining when a medication delivery device is ready for use, according to some embodiments of the technology described herein.

FIG. 10 is a flowchart showing an exemplary computerized method 1000 for determining when to begin to obtain temperature data and ambient light data, according to some embodiments of the technology described herein.

FIG. 11 is a flowchart showing an exemplary computerized method 1100 for determining when to begin to obtain temperature data and ambient light data, according to some embodiments of the technology described herein.

FIG. 12 is a block diagram depicting an exemplary system 1200 for determining when a medication delivery device is ready for use, according to some embodiments of the technology described herein.

FIG. 13 is a diagram depicting an example temperature sensing circuit 1302, according to some embodiments of the technology described herein.

FIG. 14 shows an illustrative implementation of a computer system that may be used to perform any of the aspects of the techniques and embodiments disclosed herein.

### DETAILED DESCRIPTION OF INVENTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

Provided herein are techniques for evaluating whether a medication and/or a medication delivery device is ready for use. According to some embodiments, the techniques evaluate the readiness of a medication delivery device containing a medication based on available data, such as temperature data and ambient light data associated with the medication and/or medication delivery device. For example, the medication delivery device may include one or more temperature sensor(s) configured to sense a temperature of at least a portion of the medication delivery device, a temperature of a medication contained within the medication delivery device, and/or an ambient temperature to which the medication delivery device is exposed. Additionally or alternatively, the medication delivery device may include one or more ambient light sensor(s) configured to sense an amount of light to which at least a portion of the medication delivery device and/or the medication is exposed. According to some embodiments, the techniques determine, based on the temperature data and the ambient light data, whether the temperature exceeds a temperature threshold and whether the amount of ambient light exceeds an ambient light threshold. When the temperature exceeds the temperature threshold and the amount of ambient light exceeds the ambient light threshold, the techniques, in some embodiments, output an indication that the medication delivery device is ready for use. For example, the medication delivery device may include a user feedback interface (e.g., a light emitting diode (LED), display, haptic indicator, and/or audio indicator) that notifies a user that the medication delivery device is ready to use.

The inventors have appreciated that many medication products are stored at low temperatures (e.g., below ambient room temperatures) and need to be warmed up prior to consumption. Patients therefore need to remove the medication products from storage (e.g., a freezer or refrigerator) and wait a duration of time (e.g., 30 minutes) for the product to warm up. The inventors have appreciated, however, that patients may not keep track of and/or may lose track of the amount of time the medication products have been removed from storage. For example, while a patient could simply set a timer when removing the medication product from storage, patients may forget to do so. As another example, while a patient could try to mentally keep track of the elapsed time, patients may similarly forget to do so and/or lose track of the amount of elapsed time. As a result, patients may inject the medication too early, when it is still too cold. Alternatively, patients may be overly cautious and leave the product out for too long, causing the medication to spoil, which can waste the patient's time and result in possible health consequences to the patient.

The inventors have appreciated that due to battery constraints, many medication products, such as the medication delivery devices described herein, are provided to patients in a low-power mode. Such a low-power mode does not allow for a notification method to indicate the readiness of product for consumption. Furthermore, while a patient-facing pushbutton might be used to wake the product from the low-power mode, providing such a pushbutton can be costly from a manufacturing standpoint, especially when the product is single-use and disposable. Additionally, providing such a pushbutton would require patient training and interaction, which could lead to many of the same disadvantages described above. For example, the patient may forget to press the pushbutton.

The inventors have further appreciated that, even though a medication product may be exposed to temperatures that exceed the storage temperature, the medication product may not be ready for use. For example, at the manufacturing facility, during shipment, at the pharmacy, and in transit to a patient's home, the medication product may be exposed to warmer, non-storage temperatures. Accordingly, it may not be possible to determine whether the medication product is ready for use based on the temperature of the product alone.

Accordingly, the inventors have developed techniques for evaluating and outputting an automatic indication of the readiness of a medication delivery device that address (e.g., mitigate or avoid) the above-described limitations with conventional medication products. In some embodiments, the techniques obtain temperature data from a temperature sensor and light data from a light sensor (e.g., an ambient light sensor). After determining, based on the obtained data, that a sensed temperature and a sensed amount of light each exceed corresponding thresholds, the techniques output an indication that the medication delivery device is ready for use.

In some embodiments, a processing circuit of the medication delivery device may periodically perform the techniques described herein. The processing circuit may be configured to remain in low-power mode, and only wake from the low-power mode to periodically obtain the temperature data and the ambient light data, thereby retaining battery life. Additionally or alternatively, the processing circuit may be configured to wake from low-power mode upon determining that the medication delivery device is ready for use. Accordingly, a notification method can alert the user of the readiness without relying on user intervention.

In some embodiments, by considering both a temperature and an amount of ambient light to which the medication delivery device is exposed, the techniques not only detect when the medication delivery device has been removed from low-temperature storage, but also when the medication delivery device has been removed from its packaging (e.g., a box that contains the medication delivery device). As a result, the techniques can determine whether the temperature change is associated with a patient's intended use of the medication delivery device based on whether the medication delivery device is also removed from its packaging. In some embodiments, when both conditions are met, this may indicate that the medication delivery device is ready for use. Such techniques can also avoid indicating that the medication delivery device is ready for use when it is not intended for use, such as when the user stores the medication delivery device in the fridge and is putting away groceries (e.g., such that while the medication delivery device may be exposed to light, the temperature would not increase sufficiently to wake up the medication delivery device).

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

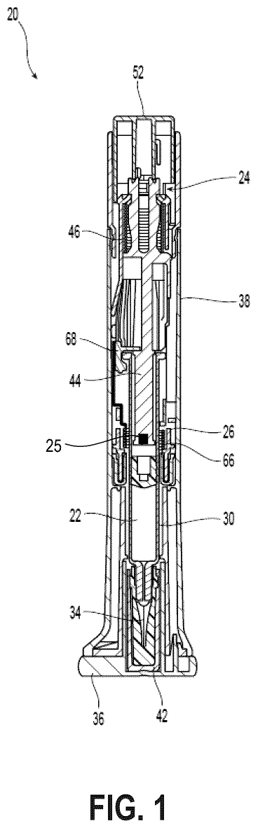

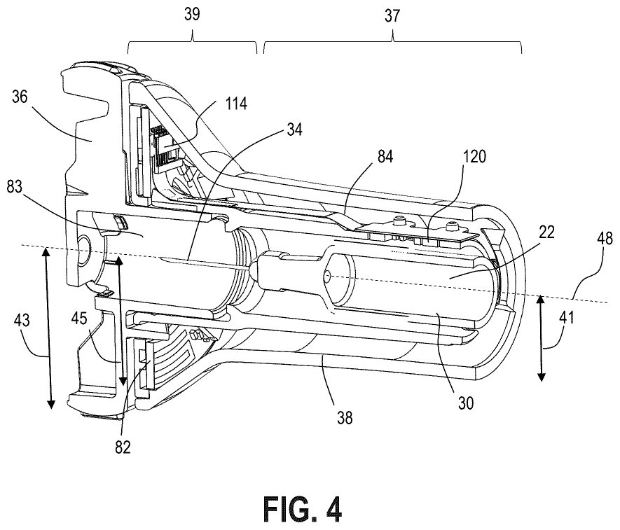

An exemplary medication delivery device or medication injection device 20 is illustrated in various operational states in FIGS. 1-3. Examples of such a device and its operation are described in U.S. Pat. No. 8,734,394 B2, entitled Automatic Injection Device with Delay Mechanism Including Dual Functioning Biasing Member, issued May 27, 2014, to Adams et. al and in U.S. Pat. No. 11,123,488 B2, entitled Status Sensing Systems within an Injection Device Assembly, issued Sep. 21, 2021, to Adams et al., the entire disclosure of both of which are hereby incorporated by reference. Device 20 includes a syringe assembly 22, a drive mechanism 24, and a retraction mechanism 26, and may include one or more main printed circuit boards (PCBs) 82 and/or one or more secondary PCBs 84 shown later, for example, in FIG. 4. Syringe assembly 22 includes a barrel 30 forming a container body for holding a medication, and a piston 32 disposed within the barrel 30 for driving the medication outside the barrel. Syringe assembly 22 also includes a needle assembly 33 having a hollow injection needle 34 and a needle hub 35 which mounts needle 34 to syringe barrel 30. Advancing piston 32 within barrel 30 toward needle 34 dispenses medication through needle 34. Although device 20 is described as an auto-injector in detail herein, other delivery devices with electronics may incorporate one or more of the various aspects described herein, such as injection pens, other kinds of auto-injectors, large volume bolus injectors, and other kinds of disposable or reusable devices configured for variable and/or fixed dosing treatment.

Devices described herein, such as device 20, may further comprise a medication, such as for example, within the syringe barrel 30. In another embodiment, a system may comprise one or more devices including device 20 and a medication. The term “medication” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, IL-2 antibody analogs or derivatives, therapeutic agents for pain-related treatments, such as galcanzeumab or lasmiditan, and any therapeutic agent that is capable of delivery by the devices described herein. The medication as used in the device may be formulated with one or more excipients. The device is operated in a manner generally as described above by a patient, caregiver or healthcare professional to deliver medication to a person.

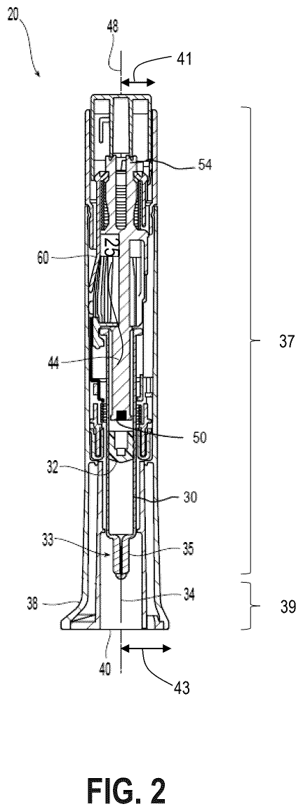

FIG. 1 illustrates device 20 in its initial, pre-use configuration. Here, an end cap 36 is secured to an injection device housing 38 and covers a proximal end opening 40 in housing 38. As used herein, distal and proximal refer to axial locations relative to an injection site when the apparatus is oriented for use at such site, whereby, for example, proximal end of the housing refers to the housing end that is closest to such injection site, and distal end of the housing refers to the housing end that is farthest from such injection site. Housing 38 may be formed from a plastic material and is shown extending generally longitudinally between a distal end in close proximity to an actuating button 52 and a proximal end in close proximity to the proximal end opening 40 along a longitudinal axis 48. As shown in FIGS. 2 and 4, housing 38 may comprise a user-graspable portion 37 configured to be grasped by a hand of a user, the user-graspable portion 37 extending a radial distance 41 outward from longitudinal axis 48. In some embodiments, the radial distance 41 may be between 5-10 mm in length (e.g., in some embodiments, 5-8 mm may be a suitable length). Also as shown in FIGS. 2 and 4, housing 38 may also optionally comprise an outwardly-flared end portion 39 at a proximal end of the housing adjacent the proximal opening 40. The optional end portion extends a radial distance 43 outward from longitudinal axis 48 that is greater than the radial distance 41. In some embodiments, the radial distance 43 may be greater than 10 mm in length. For example, in some embodiments, the radial distance 43 may be between 10-20 mm in length (e.g., in some embodiments, 15-20 mm may be a suitable length). End portion 39 may slope smoothly radially outward from the user-graspable portion 37, as shown in FIGS. 1-3. In other embodiments, end portion 39 may take the form of other shapes. For example, end portion 39 may take on any shape that extends a radial distance 43 away from longitudinal axis 48 that is greater than the radial distance 41 of the user-graspable portion.

A needle guard 42 is mounted on syringe assembly 22 and covers and surrounds needle 34. End cap 36 and needle guard 42 protect the user from accidental needle pricks and also protect needle 34 from damage. When using device 20 to dispense medication, for example, injecting the medication into a patient, end cap 36 and needle guard 42 are first removed. FIG. 2 illustrates device 20 after removal of end cap 36 and needle guard 42 from syringe assembly 22, wherein the syringe assembly is in a storage position and device 20 is ready for a dispensing event.

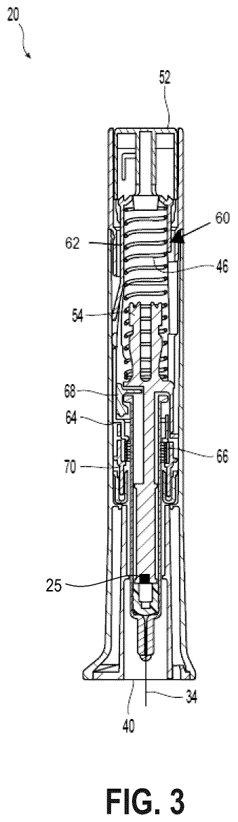

Syringe assembly 22 is moveable relative to the injection device 20 between a storage position and an injection position. FIG. 3 illustrates device 20 after the syringe assembly 22 has been moved relative to device 20 to an injection position from its storage position that is shown in FIG. 2. In the storage position (FIGS. 1 and 2), needle 34 is retracted to a position such that needle 34 is disposed within housing 38 of device 20. In the injection position (FIG. 3), needle 34 projects outwardly from housing 38 beyond proximal opening 40 in the proximal direction parallel to longitudinal axis 48 whereby needle 34 may be inserted into a patient.

Drive mechanism 24 includes a plunger 44 which engages piston 32. Drive mechanism 24 includes a spring 46 that drives plunger 44 in a translational movement. In the illustrated embodiment, spring 46 advances plunger 44 along a linear path defined by the longitudinal axis 48 of device 20. As plunger 44 is advanced, foot 50 of plunger 44 contacts piston 32. As the plunger 44 is further advanced, syringe assembly 22 is advanced along axis 48 from its storage position to its injection position. After advancement of syringe assembly 22 to its injection position, the continued proximal advancement of plunger 44 advances piston 32 proximally within barrel 30 from its initial piston position (shown in FIGS. 1 and 2) to its final piston position (shown FIG. 3) to cause medication to be dispensed from needle 34 in a dispensing event. Prior to any dispensing of medication and when syringe barrel 30 holds the full original volume of medication, piston 32 will be in its initial piston position. After advancing piston 32 the full extent of its travel length toward needle assembly 33, piston 32 will be in its final piston position proximate needle assembly 33 and the medication from within barrel 30 will have been discharged. In a single use, syringe assembly 22 will hold a single dose of medication which will be delivered in a single injection event and piston 32 will be advanced from its initial piston position to its final piston position in that single injection event to thereby deliver the entire single dose contents of syringe assembly 22. While the device is shown as a single use device, device 20 may also be configured as a multiple-use device with appropriate modifications.

The advancement of plunger 44 will generally not result in the dispensing of medication from syringe assembly 22 until after syringe assembly 22 has been advanced to the injection position. There are factors that may inhibit the medication from being dispensed before the syringe is advanced to the injection position. A factor may be the friction between piston 32 and barrel 30. Typically, piston 32 will be formed out of a rubber material and barrel 30 will be glass. The frictional resistance between these two components may be sufficient to prevent the advancement of piston 32 within barrel 30 until syringe assembly 22 is advanced to its injection position and engagement with a suitable stop member prevents the further advancement of syringe assembly 22. Additionally, the medication within the syringe may be somewhat viscous and thereby somewhat resistant to flowing out of needle 34. If necessary, modification of piston 32 and syringe barrel 30 to alter the frictional resistance of dispensing motion of the engagement member 32 relative to syringe barrel 30 may limit or prevent the premature dispensing of medication before container 22 reaches its injection position.

To activate drive mechanism 24, a person depresses actuating button 52 at the distal end of device 20. Depressing button 52 disengages one or two elongate prongs 54 on plunger 44 from a shuttle assembly 60 thereby allowing spring 46 to axially advance plunger 44. Spring 46 has a helical shape and surrounds prongs 54. The proximal end of spring 46 biasingly engages a flange on plunger 44.

Shuttle assembly 60 may include an upper shuttle member 62 and a lower shuttle member 64. Shuttle members 62, 64 are fixed together in the final assembly. In the final assembly, upper shuttle member 62 captures button 52 and spring 46 limiting the axial movement of these parts in the distal direction. Prongs 54 engage surfaces on upper shuttle 62 when the device is in the condition shown in FIGS. 1 and 2. Depressing button 52 causes tabs on button 52 to engage ramps on prongs 54 to bias prongs 54 inwardly to disengage prongs 54 from upper shuttle member 62. After prongs 54 have been disengaged, spring 46 exerts a biasing force on the flange to advance plunger 44 from the position shown in FIG. 2 to the position shown in FIG. 3. As plunger 44 is advanced, it moves syringe assembly 22 to the injection position and then advances piston 32 to dispense medication as discussed above.

After the dispensing event is complete, retraction mechanism 26 optionally moves syringe assembly 22 from the injection position shown in FIG. 3 back to a retracted position. More specifically, the retraction mechanism is adapted to move the medication container from the injection position to the retracted position in a retraction movement. The retracted position may be similar to the storage position in that the syringe assembly is drawn back into the housing 38 such that needle 34 no longer projects proximally from proximal opening 40 and is disposed entirely within housing 38. In some embodiments, the retracted position may be the same as the storage position. In other embodiments, however, a syringe assembly 22 in the retracted position may be located slightly proximal or distal to a syringe assembly in the storage position. In the illustrated embodiment, the retraction mechanism includes a spring 66, a syringe carrier 68 and a rotary member 70 that acts as a follower. In yet other embodiments, the device 20 may include no retraction mechanism 26 such that the syringe assembly remains in its injection position indefinitely after the medication has been dispensed, until the syringe assembly is manually removed or repositioned by a user.

Plunger 44 may include an outrigger (not shown) which unlocks rotary member 70 as plunger 44 nears the end of its travel in the proximal direction. Rotary member 70 is rotationally secured to lower shuttle member 64 by engagement between a latch and a latching recess in lower shuttle member 64. The outrigger unlocks member 70 by depressing the latch. Spring 66 is torsionally preloaded and has one end engaged with member 70 and an opposite end engaged with shuttle assembly 60. Upon depression of the latch, spring 66 causes member 70 to rotate.

Member 70 is rotatable within housing 38 but is not axially moveable relative to housing 38. Other embodiments may include a member 70 that is also axially movable. The rotation of member 70 serves as a delay mechanism to prevent retraction mechanism 26 from retracting syringe assembly 22 until after the syringe assembly has finished delivering its dose of medication. The speed of rotation of member 70 may be adjusted by adjusting a viscosity of grease disposed on or around surfaces of member 70 that are in contact with housing 38—a more viscous grease results in slower rotation, while a less viscous grease results in faster rotation. A radial flange on rotary member 70 may engage a ledge within housing member 38 to limit the proximal movement of member 70. Spring 66 may exert an axial force, torsional force, or both forces on member 70 to bias member 70 proximally to thereby maintain member 70 in an axial position where the radial flange of member 70 engages the interior ledge of housing member 38.

Shuttle assembly 60 may include axially extending channels or ribs that engage corresponding features on housing member 38 that allow shuttle assembly 60 to move axially within housing 38 but which prevent the relative rotation of shuttle assembly 60 relative to housing member 38. Shuttle assembly 60 is biased in the distal direction by spring 66 but is prevented from moving distally by engagement of a latch (not shown) before activation of drive mechanism 24. When rotary member 70 completes its rotation, it disengages the aforementioned latch, thus allowing shuttle assembly 60 to move distally under the biasing force of spring 66.

As shuttle assembly 60 moves distally, it carries syringe assembly 22 distally and moves it back to the storage position shown in FIG. 2. Spring 66 biases the retraction mechanism 26 distally and thereby maintains syringe assembly 22 in its retracted position after an injection event. A locking mechanism such as a detent on the shuttle assembly 60 and a recess on the housing 38 member may additionally provide a locking engagement to secure syringe assembly 22 in the retracted position with needle 34 disposed within housing 38 after an injection event whereby the user may then dispose or otherwise handle device 20 in a safe manner.

Although FIGS. 1-3 depict and describe an exemplary drive mechanism 24 and an exemplary retraction mechanism 26, other mechanisms may also be used to drive syringe assembly 22 from the storage position to the injection position, and/or from the injection position to the retracted position. Such drive and/or retraction mechanisms may (but need not) include one or more springs or deformable parts that store energy when they are held in a pre-triggered state and, when triggered, release said stored energy to drive the syringe assembly from the storage position to the injection position, and/or from the injection position to the retracted position. Such mechanisms may (but need not) include mechanisms that generate motive force using chemical reactions or processes, e.g., by generating gas through the mixture of two or more reagents, or by igniting a small amount of combustible or explosive material. Such chemically-driven mechanisms may comprise one or more storage containers for the chemical reagents, a trigger that punctures or opens said storage containers, allows said reagents to mix, and/or which provides a spark or other ignition source for beginning the chemical reaction, and a movable piston or other component that moves in response to increasing gas pressure generated by the resulting chemical reaction. Such mechanisms may (but need not) include mechanisms that use stored electrical power (e.g., in a battery) to run electric motors that drive and/or retract the syringe assembly, or to trigger other physical or chemical mechanisms. Such mechanisms may (but need not) include hydraulic or pneumatic systems (e.g., tubes), gears, cables, pulleys, or other known components for transferring kinetic energy from one component to another. In some embodiments, rather than having separate mechanisms for driving the syringe assembly and then retracting the syringe assembly, a single mechanism may be configured to both drive and then retract the syringe assembly.

FIG. 4 illustrates an exemplary placement of one or more main PCBs 82 within the end portion 39, according to a first set of embodiments of device 20. The one or more main PCBs may be arranged perpendicular to the longitudinal axis 48, and may be stacked on top of each other, and/or may be arranged next to each other on the same plane perpendicular to the longitudinal axis 48. The main PCB(s) define an opening 83 through which injection needle 34 of syringe assembly 22 is configured to pass, for example, when end cap 36 is removed and the injection needle is driven proximally to inject the patient during a dispensing event. As shown, the main PCB(s) extend a radial distance 45 away from the longitudinal axis 48 that is greater than the radial distance 41 of the user-graspable portion 37. FIG. 4 also shows one or more secondary PCBs 84 that extend substantially perpendicular to the main PCBs and parallel to the longitudinal axis 48-secondary PCB(s) may be communicatively coupled to the main PCB(s) via one or more PCB connectors 114. While secondary PCB(s) 84 may mount additional sensing systems, such secondary PCBs are optional and may be excluded in certain embodiments to decrease manufacturing complexity and costs.

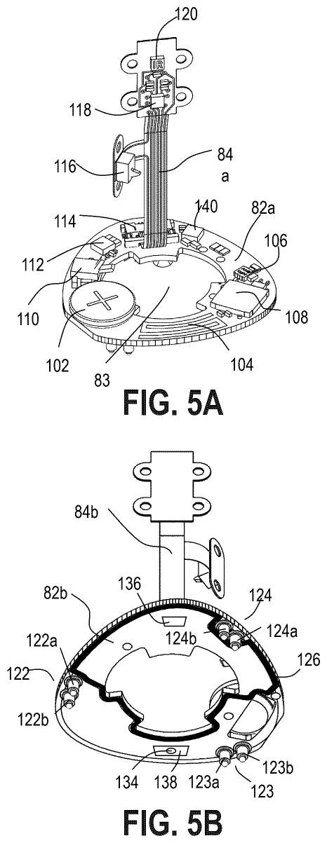

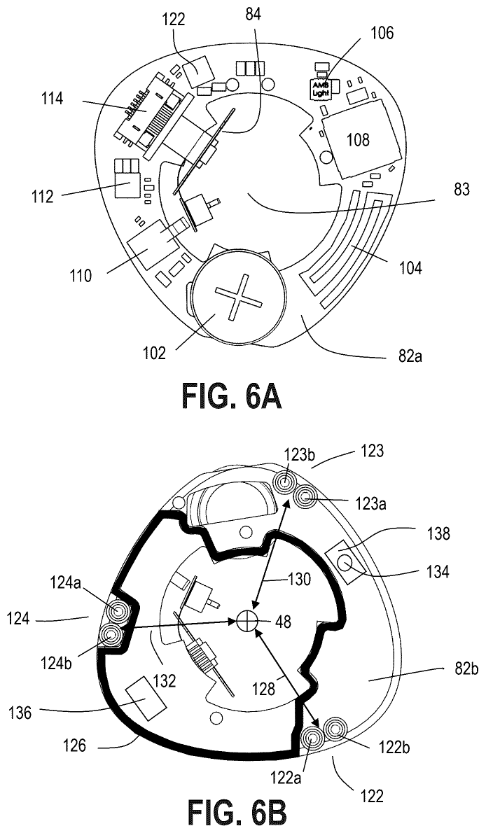

FIG. 5A shows a top perspective view of the main PCB(s) and the secondary PCB(s), while FIG. 5B shows a bottom perspective view of the same PCB(s). FIGS. 6A and 6B show a top and bottom view of the same PCBs, respectively. Main PCB(s) 82 (shown in FIG. 4) may have a top surface 82a (shown in FIGS. 5A and 6A—top surface 82a is understood to be part of PCB(s) 82) that includes or supports a power source 102 which, in some embodiments, may comprise a battery such as a coin cell battery. Power source 102 provides electrical power to the electrical components integrated or coupled with injection device 20. The main PCB(s) 82 may also include a processing circuit 108. In some embodiments, processing circuit 108 may take the form of a System on Chip (SOC) integrated circuit that includes a processor, memory, and input/output ports. However, processing circuit 108 may also be implemented using other types of components, such as a microcontroller (MCU), or an Application Specific Integrated Circuit (ASIC). Processing circuit 108 may be configured to execute computer-executable instructions stored on non-transitory storage media. Main PCB(s) may also include a plurality of different types of sensors, such as a micro-switch sensor 110, a magnetometer 112, an accelerometer 140, an ambient light sensor 106, and/or one or more skin contact resistance sensors 122, 123, and 124. In embodiments that include secondary PCB(s), the secondary PCB(s) may include further sensors, such as another micro-switch sensor 116, a magnetometer 118, and an infra-red temperature sensor 120.

Micro-switch sensors 110 and 116 may be communicatively coupled with processing circuit 108. Each micro-switch sensor may include a physical switch coupled to an electrical circuit which outputs electrical signals to processing circuit 108 depending on the physical position or orientation of the physical switch. Micro-switch sensors 110 and 116 may be used to detect the positions of components of injection device 20. For example, micro-switch sensor 110 may be used to detect whether end cap 36 is attached to the proximal end of device housing 38. As discussed in more detail below, depending on the output of micro-switch sensor 110, processing circuit 108 may indicate to a user whether end cap 36 is attached to device 20. Similarly, micro-switch sensor 116 may be used to detect whether syringe assembly 22 is in one of two states, such as (i) the storage position or (ii) the injection position. Micro-switch sensor 116 may also be configured to detect whether syringe assembly 22 is in one of three states, such as (i) the storage position, (ii) the injection position, or (iii) the retracted position. Depending on the output of micro-switch sensor 116, processing circuit 108 may indicate to the user what position syringe assembly 22 is in.

Ambient light sensor 106 may be communicatively coupled with processing circuit 108 and may detect the amount or intensity of ambient light to which injection device 20 is exposed. Over-exposure to ambient light may render medication stored in barrel 30 ineffective or unsafe for injection. In some embodiments, processing circuit 108 may log the intensity and/or duration of ambient light detected by ambient light sensor 106. If the intensity and/or duration of exposure to ambient light exceeds pre-determined thresholds, the user may be informed that the medication should not be used.

Accelerometer 140 may be communicatively coupled with processing circuit 108 and may determine the orientation of injection device 20 (e.g., pointing up, down, or sideways). This may be important for certain types of drugs which may be significantly affected by gravity due to settling of particulates, etc., which would require that the drug be delivered in a particular orientation. The processing circuit 108 may also use the output of accelerometer 140 to warn the user if the device 20 is oriented improperly for injection (e.g., if the device is upside-down).

Many types of medication need to be stored at a first, relatively cool temperature (e.g., between 36 and 46 degrees Fahrenheit, or 2 and 8 degrees Celsius) to prevent spoliation, but then need to be warmed up to a second, warmer temperature (e.g., to room temperature, or between 65 and 75 degrees Fahrenheit, or 18 and 24 degrees Celsius) before being injected into the patient's body. To ensure that the medication within barrel 30 is stored at the appropriate 40 storage temperature, and/or to ensure that the medication is warmed to the appropriate injection temperature, injection device 20 may be equipped with a mechanism for estimating the temperature of the medication. By ensuring that the medication has warmed up to the appropriate temperature, 45 this information can be transmitted to a phone, or the device itself could signal a patient that the device is ready for use. In some embodiments, this temperature-measurement function may be performed by an infra-red (IR) temperature sensor 120 on secondary PCB 84. IR sensor 120 may be 50 communicatively coupled with processing circuit 108. As best seen in FIG. 4, IR sensor 120 may be disposed adjacent to and facing towards barrel 30. IR sensor 120 may detect and measure electromagnetic radiation in the IR spectrum from barrel 30, and output an electrical signal based on the detected IR radiation. By sampling the electrical signal output by IR sensor 120, processing circuit 108 may estimate the temperature of medication within barrel 30.

Main PCB(s) may also be equipped with one or more antennas for sending or receiving wireless communications. For example, FIGS. 5A and 5B depict a Bluetooth Low Energy (BLE) antenna 104 disposed on an upper surface 82a of the main PCB(s), and a Near Field Communication (NFC) antenna 126 (shown as a thick, black-lined element) disposed on a bottom surface 82b of the main PCB(s). Other embodiments in which main PCB(s) are equipped with only one antenna, or only one type of antenna, are also possible. As discussed in further detail below, these antenna(s) may allow injection device 20 to establish a wireless communication link with an external device.

Main PCB(s) may also be communicatively coupled or integrated with a plurality of sensors that detect contact with skin tissue. Skin contact sensors may be used to verify proper contact with the user's skin before the user activates injection device 20. Injection device 20 may also indicate to the user which sensors detect skin contact and which do not; this lets the user know which way he or she should tilt or move the injection device 20 before injection. This functionality decreases the likelihood of failed injections in which the needle 34 fails to penetrate the skin of the user, or penetrates at an improperly shallow angle.

FIGS. 5B and 6B depict an exemplary embodiment that includes three skin contact sensors 122, 123, and 124 disposed on the bottom surface 82b of the main PCB(s), and arranged in a symmetrical, tri-lobed shape. In this exemplary embodiment, each skin contact sensor 122, 123, and 124 includes two separate electrical terminals-sensor 122 includes terminals 122a and 122b, sensor 123 includes terminals 123a and 123b, and sensor 124 includes terminals 124a and 124b. Although only two electrical terminals are depicted for each sensor, other embodiments in which each sensor has more than two electrical terminals are also possible. Each skin contact sensor may measure electrical resistance between its electrical terminals, and output an electrical signal based on the measured resistance to processing circuit 108. The electrical resistance of skin tissue is generally lower than that of air, and so processing circuit 108 may determine that a particular skin contact sensor is in contact with skin tissue when the measured resistance is below a predetermined threshold.

Although FIGS. 5B and 6B depict each skin contact sensor 122, 123, and 124 as having two electrical terminals, other embodiments are possible in which each skin contact sensor has only one electrical terminal. In such cases, the electrical terminal of one skin contact sensor (e.g., sensor 122) may serve as a reference electrode that outputs a predetermined voltage. The electrical terminal on each of the other two skin contact sensors (e.g., sensors 123 and 124) may serve as a sensor electrode that measures electrical resistance of a conducting path between itself and the reference electrode. When the measured resistance between the reference electrode and a particular sensor electrode is below a predetermined threshold, processing circuit 108 may determine that both the reference electrode and the particular sensor electrode are in contact with human tissue, such as skin. When both sensor electrodes (e.g., on sensors 123 and 124) report measured resistances below a predetermined threshold, processing circuit 108 may determine that the reference electrode and both sensor electrodes are in contact with human tissue.

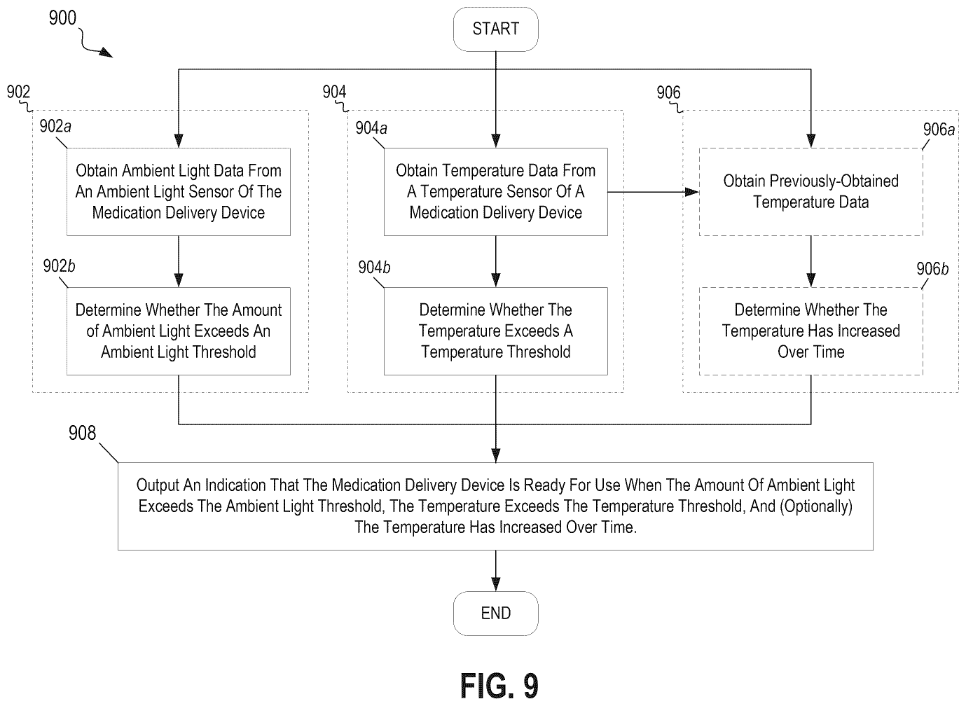

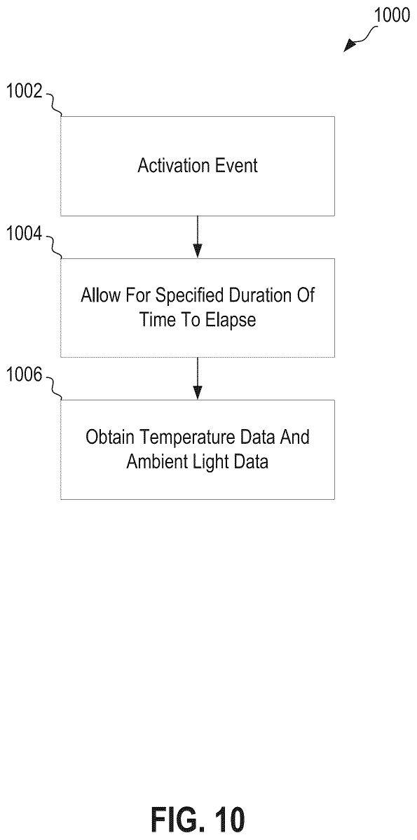

As best shown in FIG. 6B, each skin contact sensor 122, 123, and 124 may be located a radial distance 128, 130, and 132 respectively outward from the longitudinal axis 48 (which, in the view shown in FIG. 10B, extends into the page). Sensors 122, 123, and 124 may optionally be arranged to symmetrically surround the opening 83, such that radial distances 128, 130, and 132 are equal to each other, and the angular separation between each sensor is also equal (e.g., in this case, 120°). Radial distances 128, 130, and 132 are greater than radial distance 41 of the user-graspable portion 37 (as shown in FIGS. 2 and 9) and may be greater than 10 mm in length. For example, in some embodiments, radial distances 128, 130, and 132 may each be between 10 mm-20 mm in length—in some cases, a distance of 15 mm to 20 mm may be appropriate. Although three skin sensors are depicted, other embodiments having only one or two skin sensors are also possible. Conversely, embodiments with more than three skin contact sensors are also possible—in such embodiments, the skin sensors may (but need not be) arranged to symmetrically surround opening 83. For example, other embodiments comprising four to twenty skin sensors are also contemplated.

While skin contact sensors 122, 123, and 124 have been described above as measuring electrical resistance, these skin contact sensors may alternatively be configured to detect skin contact by measuring electrical capacitance. Capacitance sensors may be configured to detect proximity of human tissue by detecting such tissue's effect on an electrical field created by the sensor (e.g., by detecting the effect of such tissue on the capacitance of a circuit being monitored or measured by the sensor). Capacitance sensors do not require a metallic, electrical terminal that directly contacts skin tissue, and so may be partially or completely sealed behind a protective, non-conducting cover (e.g., made of plastic). This may increase the durability of the capacitance sensor by decreasing seepage of moisture or foreign substances into sensitive electrical components. Capacitance sensors may also reduce the danger of electrostatic discharge damaging sensitive electrical components within the device, since capacitance sensors do not require exposed metallic contacts.

Injection device 20 may also be equipped with a means for estimating the axial position or movement of piston 32 within barrel 30. This estimated axial position and/or movement may be used by processing circuit 108 to estimate the amount of medication remaining within barrel 30 and/or the amount of medication that has been dispensed, if any. In some embodiments, this may be accomplished by providing a magnet on or close to piston 32 as it slides along longitudinal axis 48, and one or more magnetometers that sense the magnetic field emitted by the magnet as it slides along the longitudinal axis. FIGS. 1-3 show an exemplary magnet 25 disposed on plunger 44 such that it maintains a fixed axial distance from piston 32 as the piston slides along longitudinal axis 48 within barrel 30. FIGS. 5A, 5B, 6A, and 6B also show exemplary placement of two magnetometers: magnetometer 112 on main PCB(s) 82, and magnetometer 118 on secondary PCB(s) 84. As shown, magnetometer 112 may be disposed radially farther from longitudinal axis 48 compared to magnetometer 118. Furthermore, magnetometer 118 may be disposed at an inter-mediate point along the length of barrel 30, instead of being positioned proximate to one end of barrel 30.

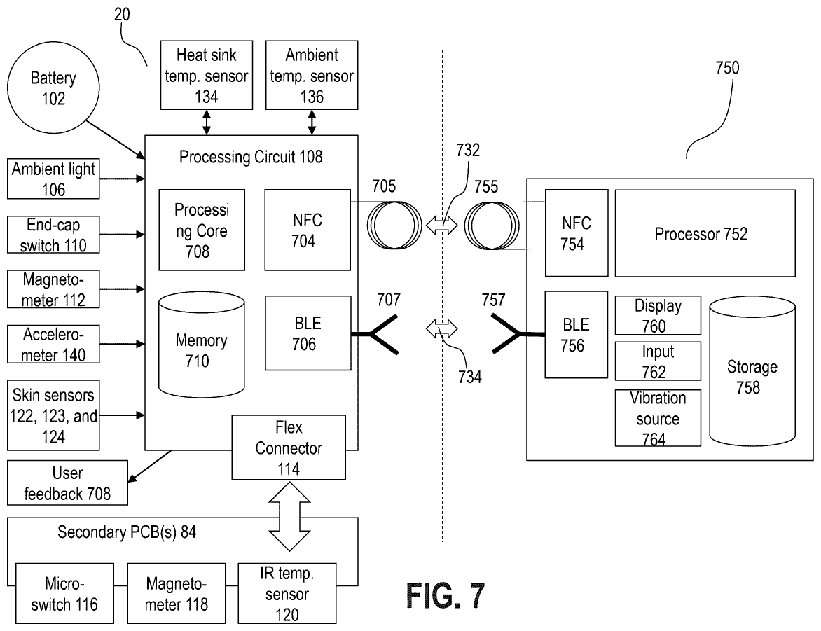

FIG. 7 provides a system architecture view of the electrical components within device 20, as well as a communication link with an exemplary external device 750. As discussed above, processing circuit 108 may be powered by a battery 102 and may comprise a processing core 708 and a memory 710 (e.g., internal flash memory, on-board electrically erasable and programmable read-only memory (EPROM), etc.). Memory 710 may store instructions that, when executed by the processing core 708, causes the processing circuit 108 to perform the operations described herein. Processing circuit 108 may also be communicatively coupled with a plurality of sensors, such as an ambient light sensor 106, end-cap micro-switch 110, magnetometer 112, accelerometer 140, and skin-contact sensors 122, 123, and 124. Processing circuit 108 may also optionally be communicatively coupled to one or more secondary PCB(s) via a flex connector 114. The secondary PCB(s) may further incorporate a micro-switch 116, magnetometer 118, and an IR temperature sensor 120. Processing circuit 108 may also be connected to a means for user feedback 708 that is integrated with device 20. The means for user feedback may include one or more indicator lights (e.g., implemented using light-emitting diodes (LEDs)), a display, a haptic indicator such as a vibration motor, and/or an auditory indicator such as a speaker). Processing circuit 108 may be communicatively coupled with each of the aforementioned components via one or more physical, electrical channels, such as (but not limited to) a General-Purpose Input/Output (GPIO) pin, an Inter-Integrated Circuit (I2C) bus, a Serial Peripheral Interface (SPI) connection, a Universal Asynchronous Receiver/Transmitter (UART) connection, and/or a Controller Area Network (CAN) bus. In some cases, signals received by the processing circuit 108 from some or all of the sensors may also be converted from an analog to a digital signal using an analog-to-digital converter (ADC).

Processing circuit 108 may also be configured to allow injection device 20 to communicate wirelessly with an external device (such as, for example, a mobile phone, a wearable device, a laptop, and/or server database). To facilitate wireless communication, processing circuit 108 may comprise a Near Field Communication (NFC) circuit 1204 communicatively coupled with an NFC antenna 705, such as NFC antenna 126 depicted in FIGS. 5B and 6B. NFC circuit 704 and NFC antenna 705 allow processing circuit 108 to establish a wireless NFC communication link 732 with an external device 750. Alternatively, or in addition, processing circuit 108 may comprise a Bluetooth Low Energy (BLE) circuit 706 communicatively coupled with a BLE antenna 707, such as BLE antenna 104 depicted in FIGS. 5A and 6A. BLE circuit 706 and BLE antenna 707 allow processing circuit 108 to establish a wireless BLE communication link 734 with external device 750.

FIG. 7 also shows an exemplary external device 750 that is physically separate from injection device 20. In this embodiment, exemplary external device 750 may take the form of a mobile smartphone having a processor 752 (e.g., a microprocessor or CPU) and storage 758. Storage 758 may comprise non-transitory computer-readable media storing computer-executable instructions that, when executed by processor 752, causes device 750 to perform the operations described herein. These computer-executable instructions may comprise a mobile application, such as a medical mobile application. Device 750 may further comprise a display 760 and a user input device 762. User input device 762 may comprise physical buttons or switches integrated with the smartphone. Although depicted separately in FIG. 7, all or a portion of user input device 762 may be integrated with display 760, e.g., in a touch-sensitive screen. Device 750 may also comprise a vibration source 764, such as a vibration motor.

Device 750 may be configured to establish a wireless communication link with injection device 20. For example, external device 750 may include an NFC circuit 754 coupled with an NFC antenna 755, which communicates with processing circuit 108 via communication link 732. Device 750 may also comprise a BLE circuit 756 coupled with a BLE antenna 707, which communicates with processing circuit 108 via communication link 734.

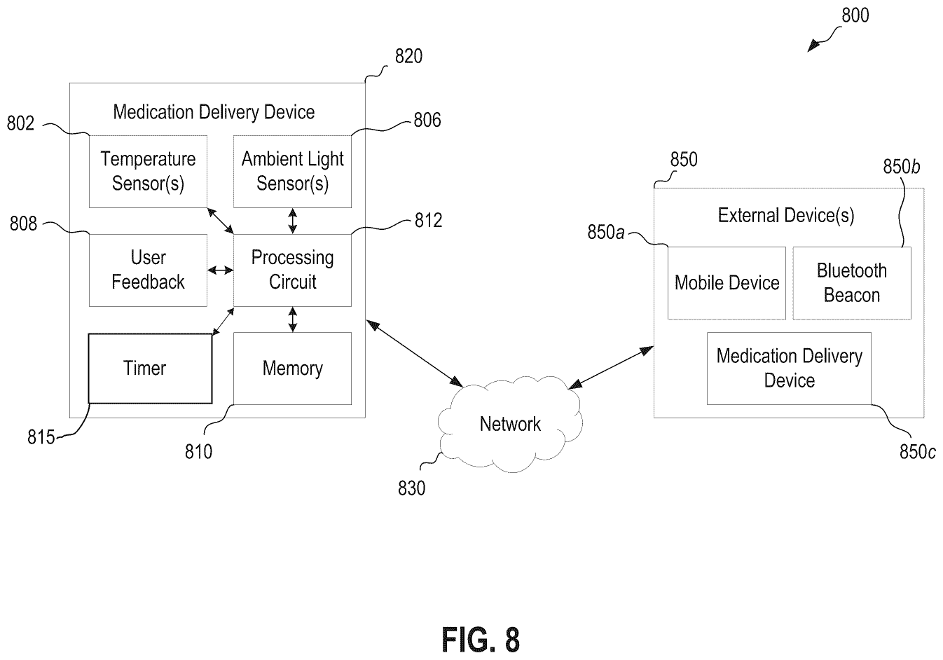

FIG. 8 is a block diagram depicting an exemplary system 800 for determining when a medication delivery device is ready for use, according to some embodiments of the technology described herein. System 800 includes medication delivery device 820, which is optionally configured to communicate with external device(s) 850 via network 830.

In some embodiments, medication delivery device 820 includes a processing circuit 812 communicatively coupled to temperature sensor(s) 802, ambient light sensor(s) 806, a user feedback interface 808, and memory 810. A timer 815 is also shown part of the device. The timer may be a clock, Real Timer Clock, a software clock, a counter, or the like. Medication delivery device 820 may include any suitable medication delivery device, such as, for example, the medication delivery device 20 described herein including at least with respect to FIGS. 1-7.

In some embodiments, the processing circuit 812 is configured to obtain temperature data from temperature sensor(s) 802 and/or ambient light data from ambient light sensor(s) 806. The processing circuit 812 processes the temperature data and/or the ambient light data to determine a state of the medication delivery device 820. For example, processing circuit 812 may process the temperature data and/or the ambient light data to determine whether the medication delivery device 820 is ready for use (e.g., to administer/consume medication).

In some embodiments, the temperature sensor(s) 802 include one or more temperature sensors each configured to sense a temperature. The sensed temperature may be a temperature of the medication delivery device 820, a temperature of a medication contained within the medication delivery device 820, and or an ambient temperature to which at least a portion of the medication delivery device 820 is exposed. The temperature sensor(s) 802 may take the form of any suitable sensor for sensing temperature, such as, but not limited to, a thermistor (e.g., a negative temperature coefficient (NTC) thermistor or a resistance temperature detector (RTD)), a thermocouple, an infrared (IR) temperature sensor, or a semiconductor-based temperature sensor. For example, the temperature sensor(s) 802 may include ambient temperature sensor 135 and/or IR temperature sensor 102, described herein including at least with respect to FIGS. 5A-7.

In some embodiments, the processing circuit 812 is configured to estimate a state of the medication delivery device 820 based on temperature data obtained from temperature sensor(s) 802 and/or memory 810. For example, the processing circuit 812 may be configured to determine, based on the temperature data, whether a temperature associated with the medication delivery device 820 exceeds a temperature threshold. Additionally or alternatively, the processing circuit 812 may be configured to determine whether the temperature associated with the medication delivery device 820 has increased over a period of time. In some embodiments, the temperature associated with the medication delivery device 820 includes a temperature of a medication held within the medication delivery device 820, an ambient temperature to which the medication delivery device 820 is exposed, and/or a temperature of the medication delivery device 820 itself.

In some embodiments, the ambient light sensor(s) 806 include one or more light sensors configured to sense an amount of light to which at least a portion of the medication delivery device 820 is exposed. The ambient light sensor(s) 806 may take the form of any suitable sensor for sensing light, such as, but not limited to, a sensor utilizing one or more photodiodes, photoresistors, and/or phototransistors. For example, the ambient light sensor(s) 806 may include ambient light sensor 106, described herein including at least with respect to FIGS. 5A-7.

In some embodiments, the processing circuit 812 is configured to estimate a state of the medication delivery device 820 based on ambient light data obtained from ambient light sensor(s) 806 and/or memory 810. For example, the processing circuit 812 may be configured to determine, based on the ambient light data, whether an amount of ambient light exceeds an ambient light threshold. In some embodiments, the ambient light includes the ambient light to which at least a portion of the medication delivery device 820 has been exposed.

In some embodiments, processing circuit 812 is configured to estimate a state of the medication delivery device 820 based on a combination of the ambient light data and the temperature data. For example, the processing circuit 812 may be configured to determine, based on the ambient light data and the temperature data, whether an amount of ambient light has exceeded an ambient light threshold, whether a temperature has exceeded a temperature threshold, and/or whether a temperature has increased over a duration of time. Techniques for estimating the state of a medication delivery device are described herein including at least with respect to FIG. 9.

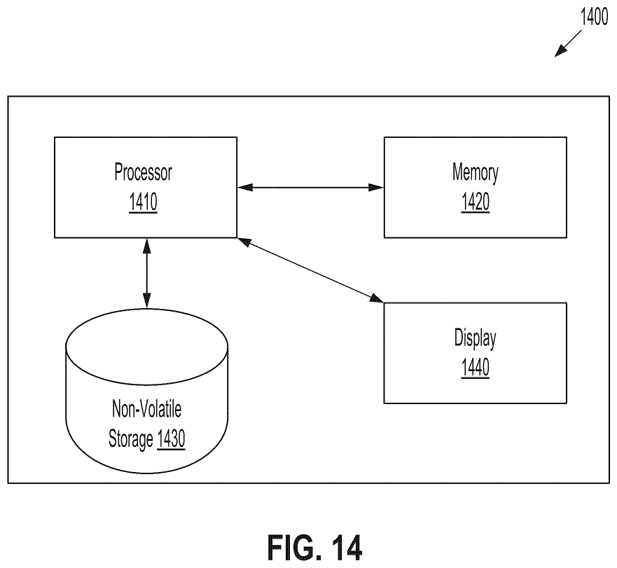

According to some embodiments, the processing circuit 812 may take the form of a processor (e.g., a microprocessor or microcontroller, field-programmable gate arrays (FPGAs) and/or digital signal processors (DSPs, or any combination of the foregoing)) configured to execute logic stored in a memory to perform the operations described herein. The term “logic”, “control logic”, “instructions” or “application” as used herein may include software and/or firmware executing on any of the aforementioned processing circuits. Further examples of processors are described herein including at least with respect to FIGS. 7 and 14.

In some embodiments, the processing circuit 812 is configured to communicate with the user feedback interface 808 and cause the user feedback interface 808 to output feedback (e.g., to a user). The user feedback interface 808 may include one or more indicator lights (e.g., implemented using light-emitting diodes (LEDs)), a display, a haptic indicator such as a vibration motor, and/or an auditory indicator such as a speaker. In some embodiments, the processing circuit 812 may cause the user feedback interface 808 to output feedback based on the state of the medication delivery device 820. For example, the processing circuit 812 may cause the user feedback interface 808 to illuminate an LED, display text and/or graphics, vibrate, and/or output a sound in response to receiving an indication from processing circuit 812 that the medication delivery device 820 is ready for use.

In some embodiments, the processing circuit 812 is configured to transmit to memory the temperature data, ambient light data, and/or data indicative of the state of the medication delivery device 820. The memory 810 may be any suitable computer readable medium that is accessible by the processing circuit and includes both volatile and non-volatile memory. Exemplary memory includes random-access memory (RAM), read-only memory (ROM), electrically erasable programmable ROM (EEPROM), flash memory, a magnetic storage device, optical disk storage, or any other suitable medium which is configured to store data, and which is accessible by the processor circuit, whether directly or indirectly via one or more intermediary devices or wired or wireless communication links. Although the preceding description assumes that the memory 810 is separate from but communicably coupled to the processing circuit, in some embodiments the memory 810 may also be integrated with the processing circuit 812. In some embodiments, instead of a processor that executes logic stored in memory 810, the processing circuit 812 may take the form of hard-wired logic, e.g., a state machine and/or an application-specific integrated circuit (ASIC) that performs the functions described herein. Further examples of memory are described herein including at least with respect to FIGS. 7 and 14.

In some embodiments, the medication delivery device 820 (e.g., the processing circuit 812) is configured to communicate with one or more of external device(s) 850 via network 830. Example external device(s) include a mobile device 850a, a Bluetooth beacon 850b, and a second medication delivery device 850c.

Mobile device 850a may comprise any device that receives, stores, and/or processes data from medication delivery device 820 via a wireless signal received by a communication circuit of the mobile device 850a. Exemplary mobile devices include a smartphone, a smartwatch, a tablet, and/or a laptop. The wireless signal may be an active signal, in which the mobile device 850a receives signals transmitted by a communication circuit (not shown) of the medication delivery device 820, or it may be a passive signal, in which mobile device 850a senses modulations to a signal transmitted by the medication delivery device 820. Mobile device 850a may include a separate communication circuit configured to communicate with other devices (e.g., using long-range or cellular transmission protocols).

In some embodiments, medication delivery device 820 is configured to transmit to the mobile device 850a information indicative of the state of the medication delivery device 820. For example, such information may indicate that the medication delivery device 820 is ready for use. In some embodiments, the mobile device 850a is configured to display, on a user interface, information indicative of the state of the medication delivery device 820. For example, a display on a user interface of the mobile device 850a may notify a user that the medication delivery device 820 is ready for use. Additionally or alternatively, the user interface of the mobile device may be configured to display additional information about the medication delivery device 820, such as information about the medication held within the medication delivery device 820, information about the patient's dosing regimen, and/or any other suitable information as aspects of the technology described herein are not limited in this respect.

In some embodiments, a Bluetooth beacon 850b comprises a hardware transmitter configured to broadcast a Bluetooth signal (e.g., a Bluetooth Low Energy (BLE) signal) to nearby devices (e.g., a medication delivery device, mobile device, etc.). In particular, the Bluetooth beacon 850b may transmit a packet of information including a unique identifier (e.g., a Universally Unique Identifier (UUID)) configured to cause an event at receiving devices. For example, the medication delivery device 820 may be configured to receive a signal from Bluetooth beacon 850b, which may cause an activation event at processing circuit 812 of the medication delivery device 820. For example, the signal may cause the processing circuit 812 to enter a low-power mode.

In some embodiments, medication delivery device 850c is a second medication delivery device, separate from medication delivery device 820. For example, medication delivery device 850c and medication delivery device 820 may be stored together (e.g., in a same freezer or refrigerator). In some embodiments, the medication delivery device 850c and the medication delivery device 820 may hold doses of medication that are to be taken together. In some embodiments, the medication delivery device 850c and medication delivery device 820 may hold doses of medication that should be taken at separate times.

In some embodiments, medication delivery device 820 and medication delivery device 850c are configured to communicate with one another via network 830. For example, the medication delivery device 850c may comprise a communication circuit that enables wireless communication with medication delivery device 820. In some embodiments, medication delivery device 850c may transmit information indicative of its state to medication delivery device 820. For example, medication delivery device 850c may transmit information that indicates it is or was previously ready for use. Medication delivery device 820 may store information received from medication delivery device 850c in memory 810 and/or transmit the information to another of the external device(s), such as mobile device 850a.

Network 830 may be or include a wide area network (e.g., the Internet), a local area network (e.g., a corporate Internet), and/or any other suitable type of network. Any of the devices shown in FIG. 8 may connect to the network 830 using one or more wired links, one or more wireless links, and/or any suitable combination thereof. Accordingly, the network 830 may be, for example, a hard-wired network (e.g., a local area network within a healthcare facility), a wireless network (e.g., connected over Wi-Fi and/or cellular networks), a cloud-based computing network, or any combination thereof.

FIG. 9 is a flowchart showing an exemplary method 900 for determining when a medication delivery device (e.g., medication delivery device 820) is ready for use, according to some embodiments of the technology described herein. Method 900 may be implemented on a medication delivery device, such as medication delivery device 820, for example, and may be a computerized method or implemented with analog.

In some embodiments, method 900 includes subprocess 902 for evaluating an amount of light to which a medication delivery device is exposed, subprocess 904 for evaluating a temperature to which the medication delivery device is exposed, and subprocess 906 for evaluating a trend in temperature to which the medication delivery device has been exposed. In some embodiments, one more of subprocesses 902, 904, and 906 may be optional. For example, subprocess 906 may be optional. Subprocesses 902, 904, and/or 906 may be performed in parallel or in any suitable order.

Subprocess 902 includes steps 902a and 902b. At step 902a, a processing circuit (e.g., processing circuit 812) obtains ambient light data from an ambient light sensor (e.g., ambient light sensor(s) 806) of the medication delivery device. The ambient light data may be indicative of an amount of light to which a portion (e.g., some, most, or all) of the medication delivery device is exposed. For example, the ambient light data may be indicative to an amount of light to which an external portion (e.g., a portion of the housing) of the medication delivery device is exposed.

At step 902b, the processing circuit determines, based on the ambient light data, whether the amount of ambient light exceeds an ambient light threshold. When the amount of ambient light exceeds the ambient light threshold, this may indicate that the medication delivery device has been exposed to a new environment. For example, it may indicate that the medication delivery device has been removed from a storage container (e.g., packaging, a freezer, etc.) and is now exposed to more light. Accordingly, in some embodiments, the ambient light threshold is configured to specify the transition between the amount of light in a storage environment and the amount of light in a non-storage environment. For example, the ambient light threshold may be a value in a range of 0 Lux to 50 Lux, and in one example, the ambient light threshold is 10 Lux. However, it should be appreciated that the ambient light threshold may depend on packaging and/or the expected storage conditions of the medication delivery device.

Subprocess 904 includes steps 904a and 904b. At step 904a the processing circuit obtains temperature data from a temperature sensor (e.g., temperature sensor(s) 802) of the medication delivery device. The temperature data may be indicative of an ambient temperature to which at least a portion of the medication delivery device is exposed, a temperature of the medication contained within the medication delivery device, and/or the temperature of the medication delivery device itself.

At step 904b, the processing circuit determines, based on the temperature data, whether the temperature exceeds a temperature threshold. In some embodiments, the medication contained within the medication delivery device needs to be stored at, or below, a particular temperature. When the temperature exceeds the temperature threshold, this may indicate that the medication delivery device has been exposed to a temperature that is greater than the prescribed storage temperature. Accordingly, in some embodiments, the temperature threshold is configured to specify the transition between a storage temperature and a non-storage temperature. As nonlimiting examples, the temperature threshold may be a value in a range of 0 C to 40 C, and in one example, the temperature threshold is 10 C. However, it should be appreciated that the temperature threshold may depend on the prescribed storage temperature of a medication contained within the medication delivery device.

Subprocess 906 includes step 906a and step 906b. At step 906a, the processing circuit may obtain previously-obtained temperature data. For example, the processing circuit may obtain the previously-obtained temperature data from memory (e.g., memory 810) on the medication delivery device. In some embodiments, the previously-obtained temperature data may be indicative of a temperature sensed by the temperature sensor(s) at one or more previous times (e.g., prior to performing method 900). In some embodiments, the processing circuit may also obtain the output of step 904a, which may include the most-recently obtained temperature data (e.g., obtained while performing method 900).

At step 906b, the processing circuit may determine, based on the previously-obtained temperature data and the temperature data obtained at step 904a, whether the temperature has increased over time. Evaluating whether the temperature has increased over time may be beneficial in determining whether the temperature changes are only temporary. For example, when a medication delivery device is transported between a freezer at a pharmacy and a freezer at a home of a patient, the medication delivery device may experience temporary temperature changes (e.g., the temperature increases and then decreases over a period of time). By contrast, when the medication delivery device is removed from the freezer and left out to thaw, the medication delivery device may experience permanent temperature changes (e.g., the temperature only increases over the period of time).

At step 908, method 900 includes outputting an indication that the medication delivery device is ready for use when the amount of ambient light exceeds the ambient light threshold, the temperature exceeds the temperature threshold, and (optionally) when the temperature has increased over time. In some embodiments, the processing circuit may communicate with a user feedback interface (e.g., user feedback interface 808) and/or an external device (e.g., mobile device 850a) to prompt the user feedback interface and/or external device to output the indication indicating that the medication delivery device is ready for use. For example, the user feedback interface may illuminate an LED, display text and/or graphics, vibrate, and/or output a sound in response to receiving an indication that the medication delivery device is ready for use. Additionally or alternatively, the external device may display text and/or graphics on a user interface, output a sound, and/or vibrate in response to receiving an indication that the medication delivery device is ready for use.

According to some embodiments, at step 908, if the medication delivery device is not ready for use, method 900 may return to subprocesses 902, 904, and 906. For example, the medication delivery device may not be ready for use if the ambient light does not exceed the ambient light threshold, the temperature does not exceed the temperature threshold, or the temperature has not increased over time.

According to some embodiments, at step 908, the medication delivery device may not ready for use, method 900 may delay outputting an indication for a period of time after the amount of ambient light exceeds the ambient light threshold and the temperature exceeds the temperature threshold, and (optionally) when the temperature has increased over time. For example, the medication delivery device may not be ready for use even if the ambient light exceeds the ambient light threshold, the temperature exceeds the temperature threshold, or the temperature has increased over time, when the manufacturer has determined that the temperature of the medication is less than the temperature of the device or ambient temperature. It is determined that a further delay prior to outputting an indication may allow the medication temperature to warm to an acceptable level. The method may continue to determine if the ambient light exceeds the ambient light threshold, if the temperature exceeds the temperature threshold, or if the temperature has increased over time. The amount of time delay may be based on many factors, such as, e.g., the medication formulation properties, the device structure, the container structure, and the like. In one example, it may be determined that fifteen-minute delay is adequate time for the medication temperature to reach an acceptable level for injection after the other conditions are met. In this case, once the conditions from subprocesses 902, 904, and 906 are satisfied, a timer (see timer 815 in FIG. 8) may start for the determined or predetermined amount of time and after the countdown timer has elapsed then the method outputting an indication occurs. User feedback of a warming-up indication of a first state, such as, for example, a first pattern of LED colors and/or sounds, sequence and frequency or nothing, during the phase of running subprocesses 902, 904, and 906, initially, is provided, a user feedback of another warming-up indication of a second state, different than the first state, for example, a second pattern of LED colors and/or sounds, sequence and frequency is provided, or both warming-up indications may be provided.

In some embodiments, step 908 may check for one or more additional medication delivery devices. For example, as described herein, a patient's dosing regimen may require use of multiple medication delivery devices for a single administration. Accordingly, a medication delivery device may check (e.g., via wireless signals) whether one or more additional medication delivery devices are also within range and activated and/or undergoing activation. If a second medication delivery device is not detected, then step 908 may not indicate that the medication delivery device is for use and/or may provide an associated notification to indicate that a second companion medication delivery device has not been detected within range.

As described above, the processing circuit of a medication delivery device may be configured to periodically perform method 900 to determine whether the medication delivery device is ready for use. In some embodiments, to initiate the periodic performance of method 900, the processing circuit is activated during a manufacturing step. For example, the processing circuit may be activated at some point prior to the packaging of the medication delivery device. However, after such a manufacturing step, the medication delivery device may remain in a manufacturing facility for a period of time (e.g., one or more hours, days, or weeks). While at the manufacturing facility, the medication delivery device may be packaged, repackaged, transferred to a vehicle, and handled in other ways. During this time, the medication delivery device may be exposed to temperatures and ambient light that satisfy the criteria described with respect to FIG. 9. Accordingly, the medication delivery device may be improperly awoken from the low-power mode prior to shipment to the pharmacy or patient.

Accordingly, in some embodiments, techniques are employed to delay the performance of method 900 for determining whether the medication delivery device is ready for use. FIG. 10 is a flowchart showing an exemplary method 1000 for determining a timing for obtaining temperature data and ambient light data (e.g., beginning performance of method 900), according to some embodiments of the technology described herein.

At step 1002, the processing circuit of the medication delivery device is activated. For example, the medication delivery device may be activated during a manufacturing step of the medication delivery device, as described above.

However, rather than immediately performing method 900 (e.g., obtaining and processing temperature data and ambient light data), the processing circuit is configured to allow a specified duration of time to elapse at step 1004. For example, the processing circuit may be configured to allow enough time to elapse such that it is no longer at the manufacturing facility when method 1000 proceeds to act 1006. For example, the processing circuit may be configured to allow for one or more hours, one or more days, and/or one or more weeks to elapse until method 1000 proceeds to step 1006.

At step 1006, after the specified duration of time has elapsed, the processing circuit of the medication delivery device begins to obtain temperature data and/or ambient light data (e.g., begins performing method 900).

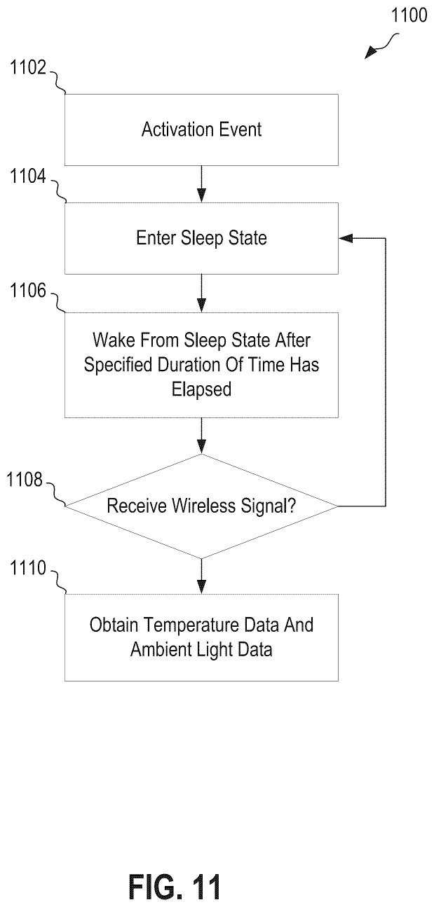

In some embodiments, additional or alternative techniques may be employed to delay the performance of method 900. FIG. 11 is a flowchart showing an exemplary method 1100 (computerized or analog) for determining a timing for obtaining temperature data and ambient light data, according to some embodiments of the technology described herein.

At step 1102, the processing circuit of the medication delivery device is activated. For example, the medication delivery device may be activated during a manufacturing step of the medication delivery device, as described above.

Following activation, in some embodiments, the processing circuit is configured to enter a sleep state at step 1104. For example, in the sleep state, the processing circuit may not perform steps of method 900, such as obtaining temperature data and ambient light data.

At step 1106, after a specified duration of time has elapsed, the processing circuit is configured to cause the activation event, such as, wake the circuit from the sleep state. For example, the processing circuit may be configured to wake from the sleep state after one or more hours, one or more days, or one or more weeks.

In some embodiments, after the processing circuit wakes from the sleep state at step 1106, the processing circuit may be configured to listen for a wireless signal. In some embodiments, the wireless signal may include any suitable wireless signal, such as, for example, a wireless signal from a Bluetooth beacon (e.g., Bluetooth beacon 850b) located in a manufacturing facility.

If, at step 1108, the processing circuit receives a wireless signal, the method 1110 returns to step 1104, and the processing circuit reenters the sleep state. For example, the processing circuit may cause another activation event, such as, reenter the sleep state for the same specified duration of time as described above with respect to act 1106. Alternatively, the processing circuit may reenter the sleep state for a different specified duration of time (e.g., one or more hours, days, or weeks).

If, at step 1108, the processing circuit does not receive a wireless signal at step 1108, method 1100 proceeds to act 1110. At act 1110, in some embodiments, the processing circuit begins to obtain temperature data and/or ambient light data (e.g., begins to perform method 900.

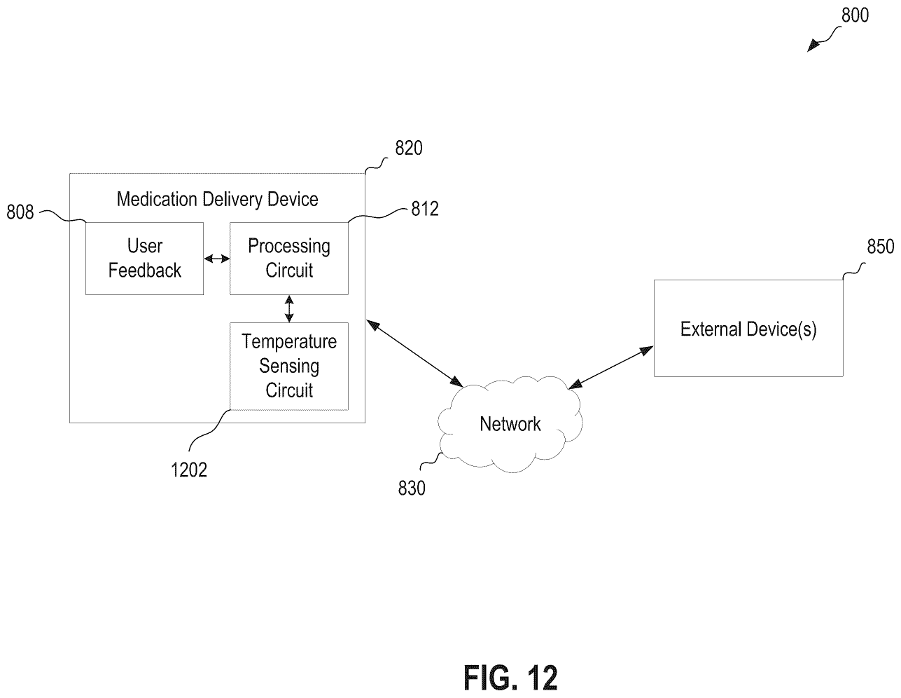

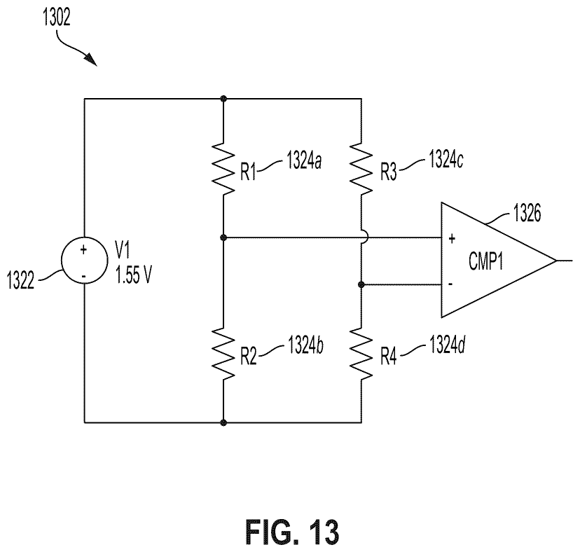

In some embodiments, additional or alternative techniques are provided for evaluating when a medication delivery device is ready for use. The techniques described herein, including at least with respect to FIGS. 12 and 13 rely on ultra-low power analog circuitry to evaluate when the medication delivery device is ready for use. In some embodiments, as described herein, the output signal of such circuitry is provided as input to a processing circuit of the medication delivery device. The signal may, in some embodiments, cause the processing circuit to wake up from the low-power mode and allow for the medication delivery device to output an indication that it is ready for use.

FIG. 12 is a block diagram depicting an exemplary system 1200 for determining when a medication delivery device is ready for use, according to some embodiments of the technology described herein. Like system 800, system 1200 includes medication delivery device 820 comprising at least processing circuit 812 and user feedback interface 808. In some embodiments, medication delivery device 820 is also optionally coupled to external device(s) 850 via network 830.

Unlike system 800, in the embodiment shown in FIG. 12, the medication delivery device 820 includes temperature sensing circuit 1202. In some embodiments, the temperature sensing circuit 1202 is configured to sense a temperature associated with the medication delivery device 820. For example, the sensed temperature may include a temperature of the medication delivery device 820 and/or an ambient temperature to which at least a portion of the medication delivery device 820 is exposed. For example, the temperature sensing circuit may comprise the example temperature sensing circuit described herein including at least with respect to FIG. 13.

In some embodiments, the temperature sensing circuit 1202 is configured to generate an output signal (e.g., output voltage) based on the sensed temperature. For example, temperature sensing circuit 1202 may provide an output signal in response to sensing a temperature that exceeds a temperature threshold.

In some embodiments, the temperature sensing circuit 1202 is configured to provide its output as input to the processing circuit 812. For example, the output of the temperature sensing circuit 1202 may serve as input to a wakeup pin coupled to the processing circuit 812. Additionally or alternatively, the output of the temperature sensing circuit 1202 may directly power the processing circuit 812. Accordingly, in some embodiments, the processing circuit 812 may not be powered (e.g., it may be in an “off” or “powered down” state) until the temperature sensing circuit 1202 provides an output signal.

In some embodiments, after the processing circuit 812 is powered on, the processing circuit 812 may communicate with user feedback interface 808 to cause the user feedback interface 808 to output feedback. For example, the processing circuit 812 may cause the user feedback interface 808 to provide feedback indicative of the state of the medication device 820. Such feedback may indicate to the user that the medication delivery device 820 is ready for use.

Additionally or alternatively, after the processing circuit 812 is powered on, the processing circuit may communicate with external device(s) 850 via network 830. The processing circuit 812 may cause the external device(s) 850 to notify a user that the medication is ready for injection and/or to display other information about the medication delivery device 820 and/or the medication.

FIG. 13 is a diagram depicting an example temperature sensing circuit 1302, according to some embodiments of the technology described herein. The temperature sensing circuit 1302 includes battery 1322, resistors 1324a-d, and comparator 1326. In some embodiments, the output of comparator 1326 is provided as input to a processing circuit, such as processing circuit 812 described herein including at least with respect to FIGS. 8 and 12.

In some embodiments, resistors 1324a-d include resistors having resistances that, when operating together with the battery 1322 and comparator 1326, and when exposed to a temperature within a specified temperature range, cause the comparator 1326 to switch between voltage output levels. Table 1 shows example values for components of temperature sensing circuit 1302 for causing the comparator 1326 to switch between voltage output levels when exposed to temperatures between 9° C. and 11° C. However, it should be appreciated that any suitable values may be used for causing the comparator 1326 to switch between voltage output levels when the temperature sensing circuit 1302 is exposed to any suitable temperature range, as aspects of the technology are not limited in this respect.

TABLE 1
Example values for components of the temperature sensing circuit 1302.

| Component | Example Values |
|---|---|
| Battery 1322 | 1.55 V |
| R1 1324a | 1026 kΩ - temp. dep. resistance element |
| R2 1324b | 2000 kΩ |
| R3 1324c | 3000 kΩ |
| R4 1324d | 1539 kΩ |

An illustrative implementation of a computer system 1400 that may be used to perform any of the aspects of the techniques and embodiments disclosed herein is shown in FIG. 14. The computer system 1400 may include one or more processors 1410 and one or more non-transitory computer-readable storage media (e.g., memory 1420 and one or more non-volatile storage media 1430) and a display 1440. The processor 1410 may control writing data to and reading data from the memory 1420 and the non-volatile storage device 1430 in any suitable manner, as the aspects of the invention described herein are not limited in this respect. To perform functionality and/or techniques described herein, the processor 1410 may execute one or more instructions stored in one or more computer-readable storage media (e.g., the memory 1420, storage media, etc.), which may serve as non-transitory computer-readable storage media storing instructions for execution by the processor 1410.

In connection with techniques described herein, code used to, for example, identify a patient for inclusion in a clinical trial may be stored on one or more computer-readable storage media of computer system 1400. Processor 1410 may execute any such code to provide any techniques for recognizing objects as described herein. Any other software, programs or instructions described herein may also be stored and executed by computer system 1400. It will be appreciated that computer code may be applied to any aspects of methods and techniques described herein. For example, computer code may be applied to interact with an operating system to recognize objects through conventional operating system processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one-time programmable memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This allows elements to optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

Various aspects are described in this disclosure, which include, but are not limited to, the following aspects:

1. A method, including: obtaining, from a temperature sensor of a medication delivery device, temperature data indicative of a temperature; obtaining, from an ambient light sensor, ambient light data indicative of an amount of ambient light to which at least a portion of the medication delivery device is exposed, wherein the medication delivery device further includes: a reservoir configured to hold the medication; and an actuating button for initiating an injection of the medication; determining, based on the temperature data, whether the temperature exceeds a temperature threshold; determining, based on the ambient light data, whether the amount of light exceeds an ambient light threshold; and outputting an indication that the medication delivery device is ready for use when the temperature exceeds the temperature threshold and the amount of ambient light exceeds the ambient light threshold.

2. The method of aspect 1, further including: determining, based on the temperature data and previously-obtained temperature data, whether the temperature has increased over time, wherein outputting the indication that the medication delivery device is ready for use further includes outputting the indication after determining that the temperature has increased over time.

3. The method of aspect 1, wherein the medication delivery device further includes a visual indicator, and wherein outputting the indication that the medication delivery device is ready for use includes outputting the indication via the visual indicator.

4. The method of aspect 3, wherein the visual indicator includes at least one light emitting diode (LED).

5. The method of aspect 1, wherein obtaining the temperature data and the ambient light data includes obtaining the temperature data and the ambient light data after a specified duration of time has elapsed since an activation event.

6. The method of aspect 1, wherein obtaining the temperature data and the ambient light data includes: receiving a wireless signal; and obtaining the temperature data and the ambient light data after a specified duration of time has elapsed since receiving the wireless signal.

7. The method of aspect 6, wherein the wireless signal includes a Bluetooth low energy (BLE) signal.

8. The method of aspect 1, wherein obtaining the temperature data includes obtaining data indicative of a temperature of a medication held within the medication delivery device.

9. The method of aspect 1, wherein the outputting step includes outputting the indication that the medication delivery device is ready for use after an elapsed period of time after the temperature exceeds the temperature threshold and the amount of ambient light exceeds the ambient light threshold and after a period.

10. The method of aspect 1, further including providing a first warming-up indication when the temperature does not exceed the temperature threshold and/or the amount of ambient light does not exceed the ambient light threshold, providing a second warming-up indication during timing of said elapsed period of time, or both.

11. A system including a memory storing instructions, and a processor configured to execute the instructions to perform the method of any of aspects 1-10.

12. A non-transitory computer-readable media including instructions that, when executed by one or more processors on a computing device, are operable to cause the one or more processors to execute the method of any of aspects 1-10.

13. A medication delivery device including a memory configured to store instructions, and a processor configured to execute the instructions to perform the method of any of aspects 1-10.

1. A method, comprising:
obtaining, from a temperature sensor of a medication delivery device, temperature data indicative of a temperature;
obtaining, from an ambient light sensor, ambient light data indicative of an amount of ambient light to which at least a portion of the medication delivery device is exposed, wherein the medication delivery device further comprises:
a reservoir configured to hold the medication; and
an actuating button for initiating an injection of the medication;
determining, based on the temperature data, whether the temperature exceeds a temperature threshold;
causing a processing circuit of the medication delivery device to wake up from a low-power mode when the temperature exceeds a temperature threshold;
determining, based on the ambient light data, whether the amount of light exceeds an ambient light threshold; and
outputting an indication that the medication delivery device is ready for use when the temperature exceeds the temperature threshold and the amount of ambient light exceeds the ambient light threshold.

2. The method of claim 1, further comprising:
determining, based on the temperature data and previously-obtained temperature data, whether the temperature has increased over time,
wherein outputting the indication that the medication delivery device is ready for use further comprises outputting the indication after determining that the temperature has increased over time.

3. The method of claim 1,
wherein the medication delivery device further comprises a visual indicator, and
wherein outputting the indication that the medication delivery device is ready for use comprises outputting the indication via the visual indicator.

4. The method of claim 3, wherein the visual indicator comprises at least one light emitting diode (LED).

5. The method of claim 1, wherein obtaining the temperature data and the ambient light data comprises obtaining the temperature data and the ambient light data after a specified duration of time has elapsed since an activation event.

6. The method of claim 1, wherein obtaining the temperature data and the ambient light data comprises:
receiving a wireless signal; and
obtaining the temperature data and the ambient light data after a specified duration of time has elapsed since receiving the wireless signal.

7. The method of claim 6, wherein the wireless signal comprises a Bluetooth low energy (BLE) signal.

8. The method of claim 1, wherein obtaining the temperature data comprises obtaining data indicative of a temperature of a medication held within the medication delivery device.

9. The method of claim 1, wherein the outputting step comprises outputting the indication that the medication delivery device is ready for use after an elapsed period of time after the temperature exceeds the temperature threshold and the amount of ambient light exceeds the ambient light threshold and after a period.

10. The method of claim 1, further comprising providing a first warming-up indication when the temperature does not exceed the temperature threshold and/or the amount of ambient light does not exceed the ambient light threshold, providing a second warming-up indication during timing of said elapsed period of time, or both.

11. A system comprising a memory storing instructions, and a processor configured to execute the instructions to perform the method of claim 1.

12. A non-transitory computer-readable media comprising instructions that, when executed by one or more processors on a computing device, are operable to cause the one or more processors to execute the method of claim 1.

13. A medication delivery device comprising a memory configured to store instructions, and a processor configured to execute the instructions to perform the method of claim 1.

14. The method of claim 1, further comprising receiving a wireless signal from a second medication delivery device to check to see if the second medication delivery device is also ready for use.

15. The method of claim 1, further comprising receiving a wireless signal from another device to reenter a sleep state from a wake up state.

16. A medication delivery device comprising:
a reservoir configured to hold the medication;
an actuating button configured to initiate an injection of the medication; and
a processor coupled to a temperature sensor and a memory configured to store instructions, the processor having a low power mode and a wake up mode, wherein the processor is configured to:
obtain, from the temperature sensor, temperature data indicative of a temperature, when the processor is in the low power mode;
determine, based on the temperature data, whether the temperature exceeds a temperature threshold;
cause the processor to wake up from the low-power mode to the wake up mode when the temperature exceeds a temperature threshold; and
outputting an indication that the medication delivery device is ready for use when the temperature exceeds the temperature threshold.

17. The medication delivery device of claim 16, wherein the processor is coupled to an ambient light sensor, the processor is configured to: obtain, from the ambient light sensor, ambient light data indicative of an amount of ambient light to which at least a portion of the medication delivery device is exposed; and output the indication that the medication delivery device is ready for use when the temperature exceeds the temperature threshold and the amount of ambient light exceeds the ambient light threshold.

18. The medication delivery device of claim 16, wherein the processor is configured to receive a wireless signal from a second medication delivery device to check to see if the second medication delivery device is also ready for use.

19. The medication delivery device of claim 16, wherein the processor is configured to a wireless signal from another device to reenter the low power state from the wake up state.
